(19) United States
(12) Patent Application Publication
(10) Pub. No.: US 2010/0172858 A1
(43) Pub. Date: Jul. 8, 2010

(54) COSMETIC COMPOSITION COMPRISING AT LEAST ONE POLYMER OBTAINED FROM A DIELECTROPHILIC MONOMER

(76) Inventors: Gwenaëlle Jegou, Saint Michel Sur Orge (FR); Nathalie Mougin, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/520,444

(22) PCT Filed: Dec. 20, 2007
(86) PCT No.: PCT/EP07/64359

§ 371 Date: Mar. 3, 2010

Related U.S. Application Data
(60) Provisional application No. 60/881,149, filed on Jan. 19, 2007.

(30) Foreign Application Priority Data
Dec. 20, 2006 (FR) 0655749

(51) Int. Cl. A61K 8/81; A61Q 5/12

(52) U.S. Cl. 424/70.16

(57) ABSTRACT

One subject of the present invention is a cosmetic composition for treating keratinous fibres, in particular human keratinous fibres such as the hair, which comprises at least one polymer obtained from dielectrophilic monomers. The composition of the invention makes it possible to give the hair body, bulk or volume in a long-lasting fashion. Another subject of the invention is use of the cosmetic composition for treating keratinous fibres, especially conditioning and/or dyeing keratinous fibres.

The present invention relates to a cosmetic composition for treating keratinous fibres, in particular human keratinous fibres such as the hair, comprising at least one polymer obtained from dielectrophilic monomers, to use of this composition for treating hair and also to a treatment method that uses it.

The hair is generally damaged and weakened by the action of external atmospheric agents such as the light and bad weather, and by mechanical or chemical treatments such as brushing, combing, bleaching, permanent waves and/or colouring. As a result the hair is often difficult to control, in particular it is difficult to untangle or style, and the hair, even thick hair, holds an attractive style with difficulty due to the fact that the hair lacks vitality, volume and liveliness.

Thus, to overcome this, it is now normal to use styling products which make it possible to condition the hair by giving it, in particular, body, bulk or volume.

These styling products are generally cosmetic hair compositions comprising one or more polymers which have a high affinity for the hair and which usually have the role of forming a film at its surface with a view to modifying its surface properties, especially for conditioning it.

One drawback linked to the use of these hair compositions lies in the fact that the cosmetic effects conferred by such compositions have a tendency to disappear, especially after the first shampooing.

Furthermore, it is known from document FR 2 833 489 to use electrophilic monomers that polymerize by an anionic route directly at the surface of the hair in the presence of a nucleophilic agent such as hydroxide ions (OH⁻) contained in water having a neutral pH. Thus, once deposited on the hair, these monomers form a polymer that results in a satisfactory sheathing.

However, the sheathing obtained from these compositions does not have a sufficient resistance relative to the various external attacks which the hair may undergo. Furthermore, the use of monomers to be polymerized on the hair for the purposes of polymerization is not always practical to implement and is not easily reproducible in terms of final properties of the polymer formed.

There is therefore a real need to find cosmetic compositions, especially for conditioning hair, which withstand shampooing and external attacks while keeping good cosmetic properties, that is to say giving the hair body, bulk or volume and this in a long-lasting manner.

Thus, one subject of the present invention is a cosmetic composition for treating keratinous fibres, in particular human keratinous fibres such as the hair, which comprises at least one polymer soluble in the composition and obtained from dielectrophilic monomers.

The composition of the invention makes it possible to give the hair body, bulk or volume in a long-lasting fashion.

Such a composition makes it possible, in particular, to obtain conditioning of the hair that has good resistance with respect to the various external attacks, especially with respect to fatty substances, such as sebum, and with respect to successive shampooing.

Another subject of the invention is a method for the cosmetic treatment of keratinous fibres that uses the cosmetic composition according to the invention.

Another subject of the present invention consists of use of the cosmetic composition for treating keratinous fibres, especially conditioning and/or dyeing keratinous fibres.

The polymer used in the present invention may be obtained from dielectrophilic monomers known in the art and according to polymerization processes that are themselves known from the prior art.

By way of dielectrophilic monomers, mention may especially be made of the electrophilic monomers of formula (I) below:

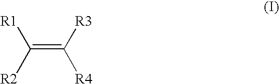

in which:
R₁ and R₂ each denote, independently of one another, a group that is not very or not at all electrophilic (also known as a group with little or no electron-withdrawing effect or little or no inductive-withdrawing effect) such as:
   a hydrogen atom;
   a linear, branched or cyclic, saturated or unsaturated hydrocarbon-based group preferably having from 1 to 20, better still from 1 to 10 carbon atoms, and optionally having one or more nitrogen, oxygen or sulphur atoms, and optionally substituted by one or more groups chosen from —OR, —COOR, —COR, —SH, —SR, —OH, and halogen atoms;
   a modified or unmodified polyorganosiloxane residue; or
   a polyoxyalkylene group;
R₃ and R₄ each denote, independently of one another, an electrophilic group (also known as an electron-withdrawing or inductive-withdrawing group) preferably chosen from —N(R)₃⁺, —S(R)₂⁺, —SH₂⁺, —NH₃⁺, —NO₂, —SO₂R, —C≡N, —COOH, —COOR, —COSR, —CONH₂, CONHR, —F, —Cl, —Br, —I, —OR, —COR, —SH, —SR, —OH groups, linear or branched alkenyl groups, linear or branched alkynyl groups, C₁-C₄ monofluoroalkyl or polyfluoroalkyl groups, aryl groups such as a phenyl group, or aryloxy groups such as a phenoxyloxy group;
R denotes a linear, branched or cyclic, saturated or unsaturated hydrocarbon-based group preferably having from 1 to 20, better still from 1 to 10 carbon atoms, and optionally having one or more nitrogen, oxygen or sulphur atoms, and optionally substituted by one or more groups chosen from —OR′, —COOR′, —COR′, —SH, —SR′, —OH, halogen atoms, or a residue of a polymer which may be obtained by radical polymerization, by polycondensation or by ring opening, R′ denoting a C₁-C₁₀ alkyl group.

The expression “electrophilic (or electron-withdrawing or inductive-withdrawing (—I)) group” is understood to mean any group that is more electronegative than carbon. Reference may be made to the work P R Wells Prog. Phys. Org. Chem., Vol. 6, 111 (1968).

The expression “group that is not very or not at all electrophilic” is understood to mean any group having an electronegativity that is less than or equal to that of carbon.

The alkenyl or alkynyl groups preferably have 2 to 20 carbon atoms, better still 2 to 10 carbon atoms.

As a linear, branched or cyclic, saturated or unsaturated hydrocarbon-based group preferably having from 1 to 20 carbon atoms, mention may especially be made of linear or branched alkyl, alkenyl or alkynyl groups, such as methyl, ethyl, n-butyl, tert-butyl, isobutyl, pentyl, hexyl, octyl, butenyl or butynyl; cycloalkyl or aromatic groups.

As a substituted hydrocarbon-based group, mention may be made, for example, of hydroxyalkyl or polyhaloalkyl groups.

As examples of unmodified polyorganosiloxanes, mention may especially be made of polyalkylsiloxanes such as polydimethylsiloxanes, polyarylsiloxanes such as polyphenylsiloxanes, polyarylalkylsiloxanes such as polymethylphenylsiloxanes.

Among the modified polyorganosiloxanes, mention may especially be made of polydimethylsiloxanes having polyoxyalkylene and/or siloxy and/or silanol and/or amine and/or imine and/or fluoroalkyl groups.

Among the polyoxyalkylene groups, mention may especially be made of polyoxyethylene groups and polyoxypropylene groups preferably having 1 to 200 oxyalkylene units.

Among the monofluoroalkyl or polyfluoroalkyl groups, mention may especially be made of groups such as —(CH₂)ₙ(CF₂)ₘ—CF₃ or —(CH₂)ₙ—(CF₂)ₘ—CHF₂ with n=1 to 20 and m=1 to 20.

The substituents R1 to R4 may optionally be substituted by a group having a cosmetic activity. The cosmetic activities particularly used are obtained from groups having dyeing, antioxidant, UV screening and conditioning functions.

By way of examples of groups having a dyeing function, mention may especially be made of azo, quinone, methine, cyanomethine and triarylmethane groups.

By way of examples of groups having an antioxidant function, mention may especially be made of groups of butylhydroxyanisole (BHA), butylhydroxytoluene (BHT) or vitamin E type.

By way of examples of groups having a UV screening function, mention may especially be made of groups of benzophenone, cinnamate, benzoate, benzylidene camphor and dibenzoylmethane type.

Among the electrophilic monomers corresponding to the formula (I), mention may be made, for example, of:
benzylidenemalononitrile derivatives of formula

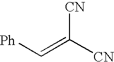

2-(4-chlorobenzylidene)malononitrile of formula

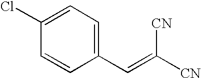

ethyl 2-cyano-3-phenylacrylate,

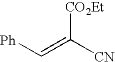

ethyl 2-cyano-3-(4-chlorophenyl)acrylate

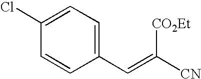

described in Sayyah, J. Polymer Research, 2000, p. 97;
methylidene malonate derivatives such as: diethyl 2-methylenemalonate described in the following references by Hopff, Makromoleculare Chemie, 1961, p. 95, De Keyser, J. Pharm. Sci., 1991, p. 67 and Klemarczyk Polymer, 1998, p. 173

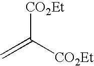

ethyl 2-ethoxycarbonylmethyleneoxycarbonyl acrylate described in the following references by Breton, Biomaterials, 1998, p. 271 and Couvreur, Pharmaceutical Research, 1994, p. 1270.

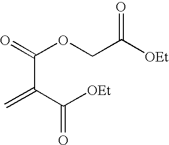

methyl α-(methylsulfonyl)acrylate (K), ethyl α-(methylsulphonyl)acrylate (L), methyl α-(tert-butyl-sulphonyl)acrylate (M), tert-butyl α-(methylsulphonyl)acrylate (N), tert-butyl α-(tert-butyl-sulphonyl)acrylate (O) derivatives by Gipstein, J. Org. Chem., 1980, p. 1486; and
1,1-bis(methylsulphonyl)ethylene (P), 1-acetyl-1-methylsulphonylethylene (Q), methyl α-(methylsulphonyl)vinylsulphonate (R), methylsulphonylacrylonitrile (S) derivatives by Shearer, U.S. Pat. No. 2,748,050:

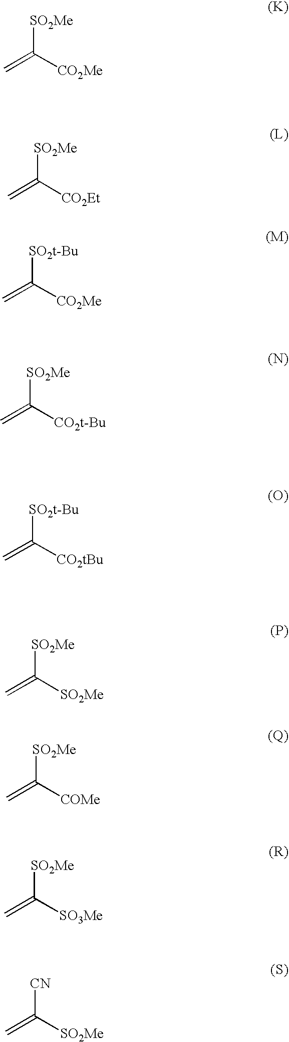

itaconimide monomers chosen from: n-butylitaconimide (F), n-(4-tolyl)itaconimide (G), n-(2-ethylphenyl)itaconimide (H), N-(2,6-diethylphenyl)itaconimide (I) especially described in the following reference by Wanatabe, J. Polymer Science: Part A: Polymer chemistry, 1994, p. 2073

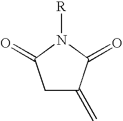

R=Bu (F), 4-tolyl (G), 2-ethylphenyl (H), 2,6-diethylphenyl (I)

According to one variant of the invention, the polymer obtained from dielectrophilic monomers is obtained from cyanoacrylate monomers. Among these monomers, mention may especially be made of the monomers from the family of cyanoacrylates and their derivatives of formula (II):

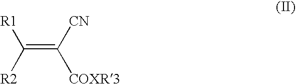

X denoting NH, S or O;
R1 and R2 having the same meanings as previously, preferably R1 and R2 representing a hydrogen atom;
R′3 representing a hydrogen atom or R such as defined in the formula (I).

Preferably, X denotes O.

By way of compounds of formula (II), mention may be made of the monomers:

a) belonging to the family of polyfluoroalkyl 2-cyanoacrylates such as:
the 2,2,3,3-tetrafluoropropyl ester of 2-cyano-2-propenoic acid of formula:

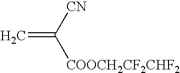

or else the 2,2,2-trifluoroethyl ester of 2-cyano-2-propenoic acid of formula:

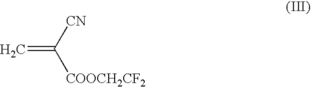

b) alkyl or alkoxyalkyl 2-cyanoacrylates:

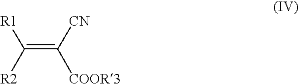

in which R′3 represents a C₁-C₁₀ alkyl or (C₁-C₄)alkoxy(C₁-C₁₀)alkyl or C₂-C₁₀ alkenyl radical.

Mention may more particularly be made of ethyl 2-cyanoacrylate, methyl 2-cyanoacrylate, n-propyl 2-cyanoacrylate, isopropyl 2-cyanoacrylate, tert-butyl 2-cyanoacrylate, n-butyl 2-cyanoacrylate, isobutyl 2-cyanoacrylate, 3-methoxybutyl cyanoacrylate, n-decyl cyanoacrylate, hexyl 2-cyanoacrylate, 2-ethoxyethyl 2-cyanoacrylate, 2-methoxyethyl 2-cyanoacrylate, 2-octyl 2-cyanoacrylate, 2-propoxyethyl 2-cyanoacrylate, n-octyl 2-cyanoacrylate and iso-amyl cyanoacrylate, allyl 2-cyanoacrylate, methoxypropyl 2-cyanoacrylate.

Within the context of the invention, it is preferred to use the monomers b). According to one preferred embodiment, the cyanoacrylate monomer or monomers are chosen from C₆-C₁₀ alkyl cyanoacrylates.

The particularly preferred monomers are octyl cyanoacrylate monomers of formula (V) and mixtures thereof:

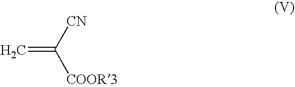

in which:
   R′3=—(CH₂)₇—CH₃;
   —CH(CH₃)—(CH₂)₅—CH₃,
   —CH₂—CH(C₂H₅)—(CH₂)₃—CH₃,
   —(CH₂)₅—CH(CH₃)—CH₃ and
   —(CH₂)₄—CH(C₂H₅)—CH₃.

The amount of polymer obtained from electrophilic monomers contained in the composition of the invention may vary over a wide range. Conventionally, the amount of this polymer is between 0.1% and 100% (pure polymer); preferably between 1% and 80%, and especially between 2% and 70%.

The polymer used in the composition of the invention comprises at least three repeat units; it may be in the form of a homopolymer or a copolymer which may be linear or branched, crosslinked or non-crosslinked; it may be random, alternating, block or gradient, or even starburst.

The composition of the invention may contain one or more polymers obtained from dielectrophilic monomers. It may also contain other units obtained from monomers other than the dielectrophilic monomers described previously, such as (meth)acrylic, (meth)acrylate or vinyl monomers.

By way of example, mention may be made of:
monomers of formula I:

H₂C═CR1(Z)ₓ(R2)ₘX (I)

in which:
R₁ is a hydrogen atom or a linear or branched hydrocarbon-based radical of CₚH₂ₚ₊₁ type, with p being an integer between 1 and 12 inclusive. In particular, R₁ may represent a methyl, ethyl, propyl or butyl radical. Preferably, R1 represents hydrogen or a methyl radical.
Z is a divalent group chosen from —COO—, —CONH—, —CONCH₃—, —OCO or —O—, —SO₂— —CO—O—CO— or —CO—CH₂—CO—. Preferably, Z is chosen from COO and CONH.
x is 0 or 1, preferably 1.
R2 is a linear, branched or cyclic, saturated or unsaturated, optionally aromatic carbon-based divalent radical having 1 to 30 carbon atoms, which may comprise 1 to 18 heteroatoms chosen from O, N, S, F, Cl, Br, Si and P.

In the radical R2, the heteroatom or heteroatoms, when they are present, may be inserted into the chain of said radical R2, or else said radical R2 may be substituted by one or more groups such as hydroxyl, —CF₃, CN, epoxy or amino (NH₂, NHR′ or NR′R″ with R′ and R″, which are identical or different, representing a linear or branched C₁-C₂₂, especially methyl or ethyl, alkyl). In particular, R2 may be or may comprise:
an alkylene radical such as methylene, ethylene, propylene, n-butylene, isobutylene, tert-butylene, n-hexylene, n-octylene, n-dodecylene, n-octadecylene, n-tetradecylene, n-docosanylene;
a phenylene (ortho, meta or para) —C₆H₄— radical optionally substituted by a C₁-C₁₂ alkyl radical optionally comprising 1 to 25 heteroatoms chosen from N, O, S, F, Si and/or P; or else a benzylene —C₆H₄—CH₂— radical optionally substituted by a C₁-C₁₂ alkyl radical optionally comprising 1 to 25 heteroatoms chosen from O, N, S, F, Si and P;
a radical of formula —CO—, —O—CO—O—, —CO—O—, —O—, —O—CO—NH—, —CO—NH—, —NHCO—, —N(R′)CO—, —NH—CO—NH—, —NR′—, epoxy, —N—CO— with R′ representing a linear or branched C₁-C₂₂ alkyl optionally comprising 1 to 12 heteroatoms chosen from O, N, S, F, Si and P;
or a mixture of these radicals;
m is 0 or 1;
X is
   (a) a guanidino or amidino group, or else
   (b) a group of formula —N(R₆)(R₇) with R₆ and R₇ representing, independently of one another, (i) a hydrogen atom, (ii) a saturated or unsaturated, optionally aromatic, linear, branched or cyclic alkyl group comprising from 1 to 18 carbon atoms, which may comprise 1 to 10 heteroatoms chosen from O, N, S, F, Si and P; or (iii) R₆ and R₇ form, with the nitrogen atom, a saturated or unsaturated, optionally aromatic, ring of formula R₆NR₇ comprising in total 5, 6, 7 or 8 atoms, and especially 4, 5 or 6 carbon atoms and/or 2 to 4 heteroatoms chosen from O, S and N; said first ring possibly being fused with one or more other saturated or unsaturated, optionally aromatic, rings each comprising 5, 6 or 7 atoms, and especially 4, 5, 6 or 7 carbon atoms and/or 2 to 4 heteroatoms chosen from O, S and N. For example, R₆ and R₇ may be chosen from hydrogen, or a methyl, ethyl, propyl, isopropyl, n-butyl, tert-butyl, isobutyl, octyl, lauryl, stearyl group. Preferably, R6 and R7 are chosen, independently of one another, from H, CH₃ and C₂H₅.

Alternatively, X may represent (c) a ring:

### R′₄NR′₅R′₆

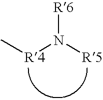

in which R′₄ and R′₅ form, with the nitrogen atom, a saturated or unsaturated, optionally aromatic, ring comprising in total 5, 6, 7 or 8 atoms, and especially 4, 5 or 6 carbon atoms and/or 2 to 4 heteroatoms chosen from O, S and N; said ring possibly being fused with one or more other saturated or unsaturated, optionally aromatic, rings each comprising 5, 6 or 7 atoms, and especially 4, 5, 6, 7 or 8 carbon atoms and/or 2 to 4 heteroatoms chosen from O, S and N; and R′₆ is chosen from H, —CH₃ and —C₂H₅.

For example, X may form a ring that may or may not be aromatic, comprising a tertiary amine group or may represent a heterocycle that may or may not be aromatic, containing a secondary or tertiary nitrogen.

Among these preferred X radicals, mention may be made of the radicals of indolyl, isoindolinyl, imidazolyl, imidazolinyl, piperidinyl, pyrazolynyl, pyrazolyl, quinoline, pyrazolinyl, pyridinyl, piperazinyl, pyrrolidinyl, quinidinyl, thiazolinyl, morpholine, guanidino, amidino radicals, and mixtures thereof.

Among the preferred monomers of formula (I), mention may be made, alone or as mixtures, of dimethylaminopropyl (meth) acrylamide, dimethylaminoethyl (meth) acrylamide, diethylaminoethyl (meth)acrylate, dimethylaminoethyl (meth)acrylate, tert-butylaminoethyl (meth)acrylate, morpholinoethyl (meth)acrylate, vinylimidazole, vinylpyridine, vinylamine, allylamine, and the above monomers in which R═H or methyl.

The ester monomers of (meth)acrylic acid of formula CH₂═CHCOOR′1 (II) or CH₂═C(CH₃)COOR′1 (II′) with R′1 representing a saturated or unsaturated, optionally aromatic (aryl, arylkyl or alkylaryl), linear, branched or cyclic carbon-based, especially hydrocarbon-based (alkyl) chain having 1 to 30 carbon atoms, optionally comprising 1 to 2 ether functional groups (—O—) and optionally 1 to 12, especially 1 to 8, functional groups chosen from —OH (hydroxy), —OR′ with R′═C₁-C₆ alkyl (alkoxy), —CN, halogen, especially Cl, F, Br or I; —CO—, SO₃H, —COOH, —OCOO—, —COO—, —OCONR—, —OCONH—, —NH—CONH—, —NR—CONR—, —CF₃, —CN, epoxy, —NHCO—, —N(R)CO— with R=linear or branched C₁-C₂₂ alkyl optionally comprising 1-12 heteroatoms.

Thus, mention may be made of methyl, ethyl, propyl, isopropyl, n-butyl, isobutyl, tert-isobutyl, hexyl, cyclohexyl 2-ethylhexyl, octyl, isooctyl, isodecyl, decyl, dodecyl, lauryl, myristyl, cetyl, palmityl, stearyl, behenyl, oleyl, tridecyl, hexadecyl, isobornyl; hydroxyethyl, hydroxypropyl; phenyl, benzyl, furfuryl; tetrahydrofurfuryl, ethoxyethyl, methoxyethyl; glycerol, 2,2,2-trifluoroethyl, and poly(ethylene-isobutylene) (meth)acrylates; and (meth) acrylonitriles.

The amide monomers of (meth)acrylic acid of formula CH₂═CHCONR′₂R″₂ (III) or CH₂═C(CH₃)CONR′₂R″₂ (III′) with R′₂, R″₂, which are identical or different, representing hydrogen or a saturated or unsaturated, optionally aromatic, linear, branched or cyclic carbon-based, especially hydrocarbon-based (alkyl) chain having 1 to 30 carbon atoms, optionally comprising 1 to 2 ether functional groups (—O—) and optionally 1 to 12, especially 1 to 8, functional groups chosen from —OH (hydroxy), —OR′ with R′═C₁-C₆ alkyl (alkoxy), —CN, halogen, especially Cl, F, Br or I; —CO—, SO₃H, —COOH, —OCOO—, —COO—, —OCONR—, —OCONH—, —NH—CONH—, —NR—CONR—, —CF₃, —CN, epoxy, —NHCO—, —N(R)CO— with R=linear or branched C₁-C₂₂ alkyl optionally comprising 1-12 heteroatoms. Thus, mention may be made of (meth)acrylamide, N-methyl(meth)acrylamide, N-isopropyl(meth)acrylamide, N-tert-butyl(meth)acrylamide, N-octyl(meth)acrylamide, N-undecyl(meth)acrylamide, N,N-dimethyl(meth)acrylamide, N,N-dibutyl(meth)acrylamide.

The vinyl ester monomers of formula CH₂═CH—OCO—R′₃ or CH₂═C(CH₃)—OCO—R′₃ with R′₃ representing a saturated or unsaturated, optionally aromatic, linear, branched or cyclic carbon-based, especially hydrocarbon-based chain having 1 to 30 carbon atoms, optionally comprising 1 to 2 ether functional groups (—O—) and optionally 1 to 12, especially 1 to 8, functional groups chosen from —OH (hydroxy), —OR′ with R′═C₁-C₆ alkyl (alkoxy), —CN, halogen, especially Cl, F, Br or I; —CO—, SO₃H, —COOH, —OCOO—, —COO—, —OCONR—, —OCONH—, —NH—CONH—, —NR—CONR—, —CF₃, —CN, epoxy, —NHCO—, —N(R)CO— with R=linear or branched C₁-C₂₂ alkyl optionally comprising 1-12 heteroatoms.

Mention may especially be made of vinyl acetate, vinyl propionate, vinyl butyrate (or butanoate), vinyl ethylhexanoate, vinyl neononanoate and vinyl neododecanoate, vinyl neodecanoate, vinyl pivalate, vinyl cyclohexanoate, vinyl benzoate, vinyl 4-tert-butylbenzoate and vinyl trifluoroacetate.

The vinyl ether monomers of formula CH₂═CHOR′4 or CH₂═C(CH₃)OR′₄ with R′₄ representing a saturated or unsaturated, optionally aromatic, linear, branched or cyclic carbon-based, especially hydrocarbon-based chain having 1 to 30 carbon atoms, optionally comprising 1 to 2 ether functional groups (—O—) and optionally 1 to 12, especially 1 to 8, functional groups chosen from —OH (hydroxy), —OR′ with R′═C₁-C₆ alkyl (alkoxy), —CN, halogen, especially Cl, F, Br or I; —CO—, SO₃H, —COOH, —OCOO—, —COO—, —OCONR—, —OCONH—, —NH—CONH—, —NR—CONR—, —CF₃, —CN, epoxy, —NHCO—, —N(R)CO— with R=linear or branched C₁-C₂₂ alkyl optionally comprising 1-12 heteroatoms.

Mention may be made of methyl vinyl ether, ethyl vinyl ether, ethylhexyl vinyl ether, butyl vinyl ether, cyclohexyl vinyl ether and isobutyl vinyl ether.

The vinyl monomers of formula CHR″5=CR5R′5 (VI) in which:

R″₅ is H or COOH, and R₅ is H, CN or COOH, and R′₅ is chosen from:
a hydrogen atom, or a group chosen from —OH, —H, —CH═O, halogen (Cl, Br, I), —COOH, —CH₂COOH, —NHC(O)H, —N(CH₃)—C(O)H, —NHC(O)CH₃, —N(CH₃)—C(O)CH₃;
a ring of formula R′₁₅NR₁₅OR′₁₅NO in which R′₁₅ and R₁₅ represent, independently of one another, H, a saturated or unsaturated, optionally aromatic, cyclic or non-cyclic, linear or branched alkyl group comprising 1 to 25 carbon atoms, optionally inserted into which are one or more heteroatoms chosen from O, N, S and P; said alkyl group possibly, in addition, being optionally substituted by one or more substituents chosen from —OH and halogen atoms (Cl, Br, I and F);
a linear or branched alkyl group comprising 1 to 25 carbon atoms;
a C₃ to C₈ cycloalkyl group such as a cyclohexane group;
a C₆ to C₂₀ aryl group such as a phenyl group;
a C₇ to C₃₀ aralkyl group, (C₁ to C₄ alkyl group) such as a 2-phenylethyl or benzyl group;
a heterocyclic group having 4 to 12 chain members containing one or more heteroatoms chosen from O, N and S;
a heterocycloalkyl group (alkyl having 1 to 4 carbons) such as a furfuryl, furfurylmethyl or tetrahydrofurfurylmethyl group, said alkyl, cycloalkyl, aryl, aralkyl, heterocyclic or heterocycloalkyl groups optionally comprising 1 to 2 ether functional groups (—O—) and optionally 1 to 12, especially 1 to 8, functional groups chosen from —OH (hydroxy), —OR′ with R′═C₁-C₆ alkyl (alkoxy), —CN, halogen, especially Cl, F, Br or I; —CO—, SO₃H, —COOH, —OCOO—, —COO—, —OCONR—, —OCONH—, —NH—CONH—, —NR—CONR—, —CF₃, —CN, epoxy, —NHCO—, —N(R)CO— with R=linear or branched C₁-C₂₂ alkyl optionally comprising 1-12 heteroatoms; and/or which may be optionally substituted by one or more linear or branched C₁-C₄ alkyl groups themselves optionally comprising 0 to 2 ether functional groups (—O—) and optionally 1 to 12, especially 1 to 8, functional groups chosen from —OH (hydroxy), —OR′ with R′═C₁-C₆ alkyl (alkoxy), —CN, halogen, especially Cl, F, Br or I; —CO—, SO₃H, —COOH, —OCOO—, —COO—, —OCONH—, —NH—CONH—, —CF₃, —CN, epoxy, —NHCO—, —N(R)CO— with R=linear or branched C₁-C₂₂ alkyl optionally comprising 1-12 heteroatoms.

Examples of such vinyl monomers are vinyl alcohol, vinylcyclohexane; vinylpyrrolidone, vinylcaprolactam, N-vinyl acetamide, N-vinylformamide, N-methyl-N-vinylformamide, N-vinylacetamide; N-methyl-N-vinylacetamide; styrene, methylstyrene, 4-tert-butylstyrene, 4-acetoxystyrene, styrenesulphonic acid, 4-methoxystyrene, 3-methylstyrene; 4-methylstyrene, 2-chlorostyrene, 3-chlorostyrene, 4-chlorostyrene, dimethylstyrene, 2,6-dichlorostyrene, 2,4-dimethylstyrene; 2,5-dimethylstyrene, 3,5-ethoxystyrene, 2,4-ethoxystyrene, 4-fluorostyrene; vinyl butyral; vinyl carbazol; vinyl chloride; vinyl formal; vinylidene chloride, vinylidene fluoride, 2-vinylnaphthalene; N-methylmaleimide; 1-octene, 1-butene, cis-chlorobutadiene, trans-chlorobutadiene, chlorotrifluoroethylene; cis-isoprene, trans-isoprene, 1-octadecene, butadiene, hexadecene and eicosene.

The following anionic monomers, and salts thereof: maleic anhydride, acrylic acid, methacrylic acid, crotonic acid, itaconic acid, fumaric acid, maleic acid, 2-carboxyethyl acrylate (CH₂═CH—C(O)—O—(CH₂)₂—COOH); styrenesulphonic acid, 2-acrylamido-2-methylpropanesulphonic acid, vinylbenzoic acid, vinylphosphonic acid, sulfopropyl (meth)acrylate, and from among the salts: sodium or potassium (meth)acrylate.

The following amphoteric monomers: N,N-dimethyl-N-(2-methacryloyloxyethyl)-N-(3-sulphopropyl)ammonium betaine (especially SPE from Raschig); N,N-dimethyl-N-(3-methacrylamidopropyl)-N-(3-sulphopropyl)ammonium betaine (SPP from Raschig), and 1-(3-sulphopropyl)-2-vinylpyridinium betaine (SPV from Raschig), and also 2-methacryloyloxyethylphosphoryl-choline.

The multivalent compounds comprising at least two polymerizable functional groups of vinyl, (meth)acrylic, allyl or (meth)acrylamide type, and especially the difunctional monomers such as 1,3-butanediol di(meth)acrylate; 1,6-hexanediol di(meth)acrylate, and N,N-dimethyldiallylammonium chloride.

Among the preferred non-dielectrophilic additional monomers, mention may be made of those chosen from vinyl neodecanoate, vinyl tert-butylbenzoate; vinylpyrrolidone; vinylcaprolactam, ethyl, methyl, tert-butyl or isobornyl (meth)acrylate; vinyl acetate, crotonic acid; (meth)acrylic acid, methacryoyl ethyl betaine, octylacrylamide; N-methyl-N-vinylimidazolinium chloride, 1-eicosene, tert-butylacrylamide, acrylamide, hexadecane, and mixtures thereof.

According to one embodiment of the invention, the polymer used in the present invention is obtained from at least 10% of dielectrophilic monomers, preferably at least 50% and more preferably 100% of dielectrophilic monomers.

The polymer obtained from dielectrophilic monomers may be obtained according to conventional radical or anionic polymerization methods described, for example, in “Chimie et physicochimie des polymères” (Chemistry and Physical Chemistry of Polymers) published by Dunod; Y. Gnanou, M. Fontanille, 10, 1998. The expression “radical polymerization” is understood to mean any polymerization initiated by a radical generator. The expression “anionic polymerization” is understood to mean any polymerization initiated by a nucleophile or anion, provided by a specific compound or by water.

These methods are described in detail in Patent Application WO 2003/053380 for synthesis via a nucleophilic route. The synthesis may also be carried out by a radical route by means of a radical initiator. By way of a radical initiator, mention may be made of initiators of the peroxide, azo, persulphate or redox type, or mixtures thereof. Among the peroxides, mention may be made of hydroperoxides such as benzoyl peroxide, acetyl peroxide, lauroyl peroxide or decanoyl peroxide. Mention may especially be made of the commercial peroxides especially sold by Arkema under the following names TRIGONOX 21S and TRIGONOX 141 sold by Akzo, the initiator being activated either via a photochemical route, or via a thermal route.

Mention may also be made of the derivatives such as: potassium persulphate, ammonium persulphate and sodium persulphate.

Azo derivatives such as 2,2′-azobis(amidinopropane) dihydrochloride (V50) and 4,4′-azobis(4-cyanovaleric acid) (ACVA).

When the polymer used in the composition of the invention is obtained from cyanoacrylate monomers, the synthesis of these polymers is especially described in U.S. Pat. No. 3,527,224, U.S. Pat. No. 3,591,767, U.S. Pat. No. 3,667,472, U.S. Pat. No. 3,995,641, U.S. Pat. No. 4,035,334 and U.S. Pat. No. 4,650,826.

According to one particular embodiment, the polymer used in the composition of the invention is obtained from dielectrophilic cyanoacrylate monomers of formula (I) which polymerize via an anionic route in the presence of a nucleophilic agent capable of generating a carbanion in contact with a nucleophilic agent, such as the hydroxide ions contained in water at a neutral pH. The term “carbanion” is understood to mean the chemical species defined in “Advanced Organic Chemistry, Third Edition”, by Jerry March, page 141.

The nucleophilic agent is a molecular compound, an oligomer, a dendrimer or a polymer having nucleophilic functional groups. Non-limitingly, mention may be made, as nucleophilic functional groups, of the functional groups: R₂N⁻, NH₂⁻, Ph₃C⁻, R₃C⁻, PhNH⁻, pyridine, ArS⁻, R—C≡C⁻, RS⁻, SH⁻, RO⁻, R₂NH, ArO⁻, N₃⁻, OH⁻, ArNH₂, NH₃, I⁻, Br⁻, Cl⁻, RCOO⁻, SCN⁻, ROH, RSH, NCO⁻, CN⁻, NO₃⁻, ClO₄⁻, H₂O, Ph representing a phenyl group; Ar representing an aryl group and R representing a C₁-C₁₀ alkyl group or a modified or unmodified polyorganosiloxane residue or a polyoxyalkylene group.

The composition of the invention may be in the form of a solution, dispersion or emulsion or gels.

The composition generally contains a cosmetically acceptable medium. Such a medium may comprise water, organic compounds, solvents or non-solvents such as alcohols, linear, branched or cyclic esters, ethers, ketones or a mixture thereof.

The organic solvent is, for example, chosen from: aromatic alcohols such as benzyl alcohol; liquid fatty alcohols, especially C₁₀-C₃₀ liquid fatty alcohols; modified or unmodified polyols such as glycerol, glycol, propylene glycol, dipropylene glycol, butylene glycol, butyl diglycol; volatile silicones such as cyclopentasiloxane, cyclohexasiloxane, polydimethylsiloxanes that may or may not be modified by alkyl and/or amine and/or imine and/or fluoroalkyl and/or carboxylic and/or betaine and/or quaternary ammonium functional groups, liquid modified polydimethylsiloxanes, mineral, organic or vegetable oils, alkanes and more particularly C₅ to C₁₀ alkanes; liquid fatty acids, liquid fatty esters and more particularly liquid fatty alcohol benzoates or salicylates.

The organic solvent is preferably chosen from organic oils; silicones such as volatile silicones, silicone gums or oils that may or may not be aminated and mixtures thereof; mineral oils; vegetable oils such as olive oil, castor oil, rapeseed oil, coconut oil, wheatgerm oil, sweet almond oil, avocado oil, macadamia oil, apricot oil, safflower oil, candlenut oil, camelina oil, tamanu oil, lemon oil or else organic compounds such as C₅-C₁₀ alkanes, acetone, methyl ethyl ketone, liquid esters of C₁-C₂₀ acids and of C₁-C₈ alcohols such as methyl acetate, butyl acetate, ethyl acetate and isopropyl myristate, dimethoxyethane, diethoxyethane, liquid C₁₀-C₃₀ fatty alcohols such as oleyl alcohol, liquid C₁₀-C₃₀ fatty alcohol esters such as C₁₀-C₃₀ fatty alcohol benzoates and mixtures thereof; polybutene oil, isononyl isononanoate, isostearyl malate, pentaerythritol tetraisostearate, tridecyl trimelate, the cyclopentasiloxane (14.7 wt %)/polydimethylsiloxane, dihydroxylated in alpha and omega positions (85.3 wt %) mixture, or mixtures thereof.

According to one preferred embodiment, the organic solvent is composed of a silicone or a mixture of silicones such as liquid polydimethylsiloxanes and liquid modified polydimethylsiloxanes, their viscosity at 25° C. is between 0.1 cst and 1 000 000 cst and more preferably between 1 cst and 30 000 cst.

Mention will preferably be made of the following oils:
the α,ω-dihydroxylated polydimethylsiloxane/cyclopentadimethylsiloxane (14.7/85.3) mixture sold by Dow Corning under the name DC 1501 Fluid;
the α,ω-dihydroxylated polydimethylsiloxane/polydimethylsiloxane mixture sold by Dow Corning under the name DC 1503 Fluid;
the dimethicone/cyclopentadimethylsiloxane mixture sold by Dow Corning under the name DC 1411 Fluid or that sold by Bayer under the name SF1214;
the cyclopentadimethylsiloxane sold by Dow Corning under the name DC 245 Fluid; and
the respective mixtures of these oils.

The composition may also contain hydrocarbons and especially paraffin oil, liquid petroleum, or hydrogenated polyisobutylene, isododecane, or else ISOPARs, volatile isoparaffins. Mention may also be made of the vegetable oils formed from fatty acid and polyol esters, in particular triglycerides, such as sunflower oil, sesame oil or rapeseed oil, or the esters derived from acids or alcohols such as palmitates, adipates and benzoates, especially diisopropyl adipate.

According to one particular embodiment, the composition of the invention comprises at least one polymerizable dielectrophilic monomer and at least one polymer obtained from dielectrophilic monomers. The polymerizable monomer or monomers present in the composition may be chosen from the monomers described above used for obtaining the polymer. According to one particular embodiment, the polymerizable monomer present in the composition and the monomers used in the synthesis of the polymer used in the composition of the invention are of the same nature, or even identical. By way of example of this embodiment, mention may be made of a composition comprising a polymerizable cyanoacrylate monomer and a polymer obtained from cyanoacrylate monomers.

The polymerizable dielectrophilic monomer or monomers present in the composition may be present in an amount ranging from 0.1 to 80% by weight, preferably in an amount ranging from 0.2 to 60% by weight, and especially in an amount ranging from 0.5 to 50% by weight, relative to the total weight of the cosmetic composition.

When the composition of the invention contains, in addition to the polymer used in the invention, polymerizable dielectrophilic monomers then the composition may contain a nucleophilic agent. The nucleophilic agent may also be added to the composition of the invention at the time of use.

According to this particular embodiment, the composition may in addition contain a polymerization inhibitor, in particular chosen from mineral or organic acids such as phosphoric acid, hydrochloric acid, nitric acid, benzenesulphonic or toluenesulphonic acid, sulphuric acid, carbonic acid, acetic acid, formic acid, propionic acid, benzoic acid, mono-, di- or tri-chloroacetic acid, salicylic acid, trifluoroacetic acid, octanoic acid, heptanoic acid and hexanoic acid. When it is present, the amount of polymerization inhibitor may vary from a few ppm to 30% by weight of the total weight of the composition, especially between 10 ppm and 15%.

The cosmetic composition according to the invention may comprise other conventional additives in the field of cosmetic compositions for hair treatment. As such, mention may be made of reducing agents, fatty substances, plasticizers, softeners, anti-foaming agents, moisturizers, pigments, clays, mineral fillers, UV screening agents, mineral colloids, peptizers, solubilizers, fragrances, preservatives, anionic, cationic, non-ionic or amphoteric surfactants, fixture or non-fixture polymers, polyols, proteins, vitamins, direct or oxidation dyes, pearling agents, propellants, and mineral or organic thickeners such as benzylidene sorbitol and N-acylamino acids, oxyethylenated or non-oxyethylenated waxes, paraffins, C₁₀-C₃₀ fatty acids such as stearic acid, lauric acid, C₁₀-C₃₀ fatty amides such as lauric diethanolamide, C₁₀-C₃₀ fatty alcohol esters such as C₁₀-C₃₀ fatty alcohol benzoates and mixtures thereof, and thickeners.

According to one particular embodiment, the composition of the invention is a composition for dyeing keratinous fibres which comprises the polymer obtained from dielectrophilic monomers, and at least one pigment.

Such a composition makes it possible to obtain lasting and visible colourings especially on dark hair since the surface pigment masks the natural colour of the fibre.

Furthermore, such a composition makes it possible to result in visible and highly chromatic colourings especially on a dark keratinous fibre without it being necessary to lighten or bleach the keratinous fibres and, consequently, without physical damage to the keratinous fibres.

Within the meaning of the present invention, the term “pigment” is understood to mean any organic and/or mineral entity having a solubility in water that is less than 0.01% at 20° C., preferably less than 0.0001% and that has an absorption between 350 and 700 nm, preferably an absorption with a maximum.

The pigments used in the composition according to the invention may especially be chosen from the organic and/or mineral pigments known from the art, especially those which are described in Kirk-Othmer's Encyclopedia of Chemical Technology and in Ullmann's Encyclopedia of Industrial Chemistry.

These pigments may be in the form of a powder or of a pigment paste. They may be coated or uncoated.

The pigments according to the invention may, for example, be chosen from white or coloured pigments, lakes, special effect pigments, such as pearlescent agents or glitter, and mixtures thereof.

As examples of white or coloured mineral pigments, mention may be made of titanium dioxide which may or may not be surface-treated, zirconium or cerium oxides, iron or chromium oxides, manganese violet, ultramarine blue, chromium hydrate and ferric blue. For example, the following mineral pigments may be used: Ta₂O₅, Ti₃O₅, Ti₂O₃, TiO, ZrO₂ as a mixture with TiO₂, ZrO₂, Nb₂O₅, CeO₂, ZnS.

As examples of white or coloured organic pigments, mention may be made of nitroso, nitro, azo, xanthene, quinoline, anthraquinone or phthalocyanine compounds, compounds of metal complex type, isoindolinone, isoindoline, quinacridone, perinone, perylene, diketopyrrolopyrrole, thioindigo, dioxazine, triphenylmethane or quinophthalone compounds.

In particular, the white or coloured organic pigments may be chosen from carmine, carbon black, aniline black, azo yellow, quinacridone, phthalocyanine blue, sorghum red, the blue pigments coded in the Colour Index under the references CI-42090, 69800, 69825, 73000, 74100, 74160, the yellow pigments coded in the Colour Index under the references CI 11680, 11710, 15985, 19140, 20040, 21100, 21108, 47000, 47005, the green pigments coded in the Colour Index under the references CI-61565, 61570, 74260, the orange pigments coded in the Colour Index under the references CI 11725, 15510, 45370, 71105, the red pigments coded in the Colour Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915, 75470, the pigments obtained by oxidizing polymerization of indolic or phenolic derivatives such as are described in Patent FR 2 679 771.

It is also possible to use pigment pastes made of organic pigments such as the products sold by Hoechst under the name:
Cosmenyl Yellow 10G: Pigment YELLOW 3 (CI 11710);
Cosmenyl Yellow G: Pigment YELLOW 1 (CI 11680);
Cosmenyl Orange GR: Pigment ORANGE 43 (CI 71105);
Cosmenyl Red R: Pigment RED 4 (CI 12085);
Cosmenyl Carmine FB: Pigment RED 5 (CI 12490);
Cosmenyl Violet RL: Pigment VIOLET 23 (CI 51319);
Cosmenyl Blue A2R: Pigment BLUE 15.1 (CI 74160);
Cosmenyl Green GG: Pigment GREEN 7 (CI 74260);
Cosmenyl Black R: Pigment BLACK 7 (CI 77266).

The pigments according to the invention may also be in the form of composite pigments such as are described in Patent EP 1 184 426. These composite pigments may be composed, in particular, of particles having an inorganic core, at least one binder ensuring the attachment of the organic pigments to the core, and at least one organic pigment at least partially covering the core.

The term “lake” is understood to mean dyes adsorbed onto insoluble particles, the assembly thus obtained remaining insoluble during use. The inorganic substrates onto which the dyes are adsorbed are, for example, alumina, silica, calcium sodium borosilicate, calcium aluminium borosilicate and aluminium. Among the organic dyes, mention may be made of cochenille carmine.

As examples of lakes, mention may be made of the products known under the following names: D & C Red 21 (CI 45 380), D & C Orange 5 (CI 45 370), D & C Red 27 (CI 45 410), D & C Orange 10 (CI 45 425), D & C Red 3 (CI 45 430), D & C Red 7 (CI 15 850:1), D & C Red 4 (CI 15 510), D & C Red 33 (CI 17 200), D & C Yellow 5 (CI 19 140), D & C Yellow 6 (CI 15 985), D & C Green (CI 61 570), D & C Yellow 10 (CI 77 002), D & C Green 3 (CI 42 053), D & C Blue 1 (CI 42 090).

The expression “special effect pigments” is understood to mean pigments which generally create a coloured appearance (characterized by a certain hue, a certain vividness and a certain lightness) which is not uniform and changes as a function of the conditions of observation (light, temperature, observation angles, etc.). They thereby contrast with white or coloured pigments which provide a conventional opaque, semi-transparent or transparent uniform colour.

As examples of special effect pigments, mention may be made of white pearlescent pigments such as mica covered with titanium dioxide, or with bismuth oxychloride, coloured pearlescent pigments such as mica covered with titanium dioxide and iron oxides, mica covered with titanium oxide and especially with ferric blue or chromium oxide, mica covered with titanium dioxide and with an organic pigment such as defined previously and also pearlescent pigments based on bismuth oxychloride. By way of pearlescent pigments, mention may be made of the following pearlescent agents: Cellini sold by Engelhard (Mica-TiO₂-lake), Prestige sold by Eckhart (Mica-TiO₂), Prestige Bronze sold by Eckhart (Mica-Fe₂O₃), Colorona sold by Merck (Mica-TiO₂—Fe₂O₃).

Mention may also be made of pigments with an interference effect which are not attached to a substrate, such as liquid crystals (Helicones HC by Wacker), interference holographic glitter (Geometric Pigments or Spectra f/x from Spectratek). Special effect pigments also comprise fluorescent pigments, whether these are substances which are fluorescent in daylight or which produce ultraviolet fluorescence, phosphorescent pigments, photochromic pigments, thermochromic pigments and quantum dots, sold, for example, by Quantum Dots Corporation.

Quantum dots are luminescent semiconductor nanoparticles capable of emitting, under light excitation, radiation having a wavelength between 400 nm and 700 nm. These nanoparticles are known from the literature. In particular, they may be manufactured according to the processes described, for example, in U.S. Pat. No. 6,225,198 or U.S. Pat. No. 5,990,479, in the publications which are cited therein and in the following publications: Dabboussi B. O. et al. “(CdSe)ZnS core-shell quantum dots: synthesis and characterisation of a size series of highly luminescent nanocristallites” Journal of Physical Chemistry B, Vol. 101, 1997, pp 9463-9475, and Peng, Xiaogang et al., “Epitaxial Growth of highly Luminescent CdSe/CdS core/shell nanocrystals with photostability and electronic accessibility” Journal of the American Chemical Society, Vol. 119, No. 30, pp 7019-7029.

The variety of pigments which can be used in the present invention makes it possible to obtain a rich palette of colours, and also particular optical effects such as interference, metallic effects.

According to one particular embodiment, the pigments are coloured pigments. The expression “coloured pigment” is understood to mean pigments other than white pigments.

The average size of the pigment used within the scope of the present invention is generally between 10 nm and 200 μm, preferably between 20 nm and 80 μm, and more preferably between 30 nm and 50 μm.

The pigments used in the cosmetic composition according to the invention may be surface-treated by an organic agent.

Thus, the pigments that have previously been surface-treated used within the scope of the invention are pigments which have completely or partially undergone a surface treatment of chemical, electronic, electrochemical, mechano-chemical or mechanical nature, with an organic agent such as those which are described, in particular, in Cosmetics and Toiletries, February 1990, Vol. 105, p. 53-64, before being dispersed in the composition according to the invention. These organic agents may be, for example, chosen from amino acids; waxes, for example carnauba wax and beeswax; fatty acids, fatty alcohols and derivatives thereof, such as stearic acid, hydroxystearic acid, stearyl alcohol, hydroxystearyl alcohol, lauric acid and derivatives thereof; anionic surfactants, lecithins; sodium, potassium, magnesium, iron, titanium, zinc or aluminium salts of fatty acids, for example aluminium stearate or laurate; metal alkoxides; polysaccharides, for example chitosan, cellulose and its derivatives; polyethylene; (meth)acrylic polymers, for example polymethyl methacrylates; polymers and copolymers containing acrylate units; proteins; alkanolamines; silicone-based compounds, for example silicones, polydimethylsiloxanes, alkoxysilanes, alkylsilanes, siloxysilicates; organic fluoro compounds, for example perfluoroalkyl ethers; fluorosilicone compounds.

The surface-treated pigments used within the scope of the invention may also have been treated by a mixture of these compounds and/or have undergone several surface treatments.

The surface-treated pigments used within the scope of the present invention may be prepared according to the surface-treatment techniques well known to a person skilled in the art or found as such commercially.

Preferably, the surface-treated pigments are covered by an organic layer.

The organic agent with which the pigments are treated may be deposited on the pigments by solvent evaporation, chemical reaction between the molecules of the surface agent or creation of a covalent bond between the surface agent and the pigments.

The surface treatment may thus be carried out, for example, by chemical reaction of a surface agent with the surface of the pigments and creation of a covalent bond between the surface agent and the pigments. This method is especially described in U.S. Pat. No. 4,578,266.

Preferably, an organic agent covalently bonded to the pigments will be used.

The agent for the surface treatment may represent from 0.1 to 50% by weight of the total weight of the surface-treated pigments or fillers, preferably from 0.5 to 30% by weight, and even more preferably from 1 to 10% by weight.

Preferably, the surface treatments of the pigments are chosen from the following treatments:
a PEG-silicone treatment such as the AQ surface treatment sold by LCW;
a chitosan treatment such as the CTS surface treatment sold by LCW;
a triethoxycaprylylsilane treatment such as the AS surface treatment sold by LCW;
a methicone treatment such as the SI surface treatment sold by LCW;
a dimethicone treatment such as the Covasil 3.05 surface treatment sold by LCW;
a dimethicone/trimethylsiloxysilicate treatment such as the Covasil 4.05 surface treatment sold by LCW;
a lauroyl lysine treatment such as the LL surface treatment sold by LCW;
a lauroyl lysine/dimethicone treatment such as the LL/SI surface treatment sold by LCW;
a magnesium myristate treatment such as the MM surface treatment sold by LCW;
an aluminium dimyristate treatment such as the MI surface treatment sold by Miyoshi;
a perfluoropolymethylisopropyl ether treatment such as the FHC surface treatment sold by LCW;
an isostearyl sebacate treatment such as the HS surface treatment sold by Miyoshi;
a disodium stearoyl glutamate treatment such as the NAI surface treatment sold by Miyoshi;
a dimethicone/disodium stearoyl glutamate treatment such as the SA/NAI surface treatment sold by Miyoshi;
a perfluoroalkyl phosphate treatment such as the PF surface treatment sold by Daito;
an acrylate/dimethicone copolymer and perfluoroalkyl phosphate treatment such as the FSA surface treatment sold by Daito;
a polymethylhydrosiloxane/perfluoroalkyl phosphate treatment such as the FS01 surface treatment sold by Daito;
a lauroyl lysine/aluminium tristearate treatment such as the LL-StAI surface treatment sold by Daito;
an octyltriethylsilane treatment such as the OTS surface treatment sold by Daito;
an octyltriethylsilane/perfluoroalkyl phosphate treatment such as the FOTS surface treatment sold by Daito;
an acrylate/dimethicone copolymer treatment such as the ASC surface treatment sold by Daito;
an isopropyl titanium triisostearate treatment such as the ITT surface treatment sold by Daito;
a microcrystalline cellulose and carboxymethyl cellulose treatment such as the AC surface treatment sold by Daito;
a cellulose treatment such as the C2 surface treatment sold by Daito;
an acrylate copolymer treatment such as the APD surface treatment sold by Daito; and
a perfluoroalkyl phosphate/isopropyl titanium triisostearate treatment such as the PF+ITT surface treatment sold by Daito.

The pigment or pigments are generally present in the composition in total amounts that are generally between 0.05 and 50% by weight of the total weight of the composition, preferably between 0.1 and 35% by weight, even more preferably between 0.5 and 20% by weight.

The composition according to the present invention may additionally comprise one or more nonsurface-treated fillers.

The composition according to the present invention may additionally comprise one or more nonsurface-treated pigments.

According to one preferred embodiment, the composition of the invention is an anhydrous composition, that is to say containing less than 1% by weight of water relative to the total weight of the composition.

According to another embodiment, it contains water and one or more liquid organic solvents.

The organic solvent or solvents of the composition generally represent from 0.01 to 99%, preferably from 50 to 99% by weight relative to the total weight of the composition.

The compositions may be in various dosage forms such as a lotion, an aerosol mousse, a conditioner or a shampoo, a gel or a wax. The compositions may be contained in a pump dispenser or an aerosol spray. After application to the hair, the compositions of the invention may be rinsed out or left on.

When the composition is contained in an aerosol, it may contain a propellant. The propellant consists of compressed or liquefied gases normally used for preparing aerosol compositions. Preferably air, carbon dioxide, compressed nitrogen or else a soluble gas such as dimethyl ether, halogenated hydrocarbons (fluorocarbons in particular) or non-halogenated hydrocarbons (butane, propane, isobutane) and mixtures thereof will be used.

Another subject of the invention is a method for treating keratinous fibres which consists in applying the composition of the invention to the fibres. When the composition also contains a polymerizable dielectrophilic monomer then it is necessary, in order to initiate the polymerization of the latter, to use a nucleophilic agent either simultaneously or during a post-treatment.

According to one variant, the method for treating keratinous fibres is a hair dyeing method which consists in applying the composition of the invention containing a pigment.

The method of the invention may comprise additional intermediate or final steps such as the application of a cosmetic product, a rinsing step, a drying step. The drying may be carried out under a hood dryer, using a hairdryer and/or using a straightening iron. In particular, the application of the compositions according to the invention may be followed by rinsing.

Another subject of the invention is a multi-compartment device or kit comprising a first compartment, comprising a composition containing at least one polymerizable dielectrophilic monomer such as defined previously and optionally at least one anionic and/or radical polymerization inhibitor, and a second compartment, comprising a second composition containing one or more polymers obtained from a dielectrophilic monomer, one of the two or both compositions possibly containing one or more cosmetic additives such as defined previously.

The examples which follow are intended to illustrate the invention without however being limiting.

### EXAMPLES

In the examples which follow, the reactants used were:
Cyclomethicone which was a cyclopentadimethylsiloxane sold by Dow Corning under the name DC 245 Fluid (D5).
2-octyl cyanoacrylate which was RITE LOK CON895, sold by Chemence;
TRIGONOX 21S (T21S) which was tert-butyl peroxy-2-ethylhexanoate [CAS number 3006-82-4], sold by Akzo Nobel;
KPS which was potassium persulphate sold by Aldrich; and
THF which was tetrahydrofuran sold by Fluka.

### Example 1

### Synthesis of poly(2-octyl cyanoacrylate), Initiation by Anionic (Water) Route—Polymer P1

Introduced into a round-bottomed flask equipped with a magnetic stirrer bar were 1 g of octyl cyanoacrylate followed by 9 g of cyclomethicone (D5). The mixture was stirred for around 1 minute. 7.25 g of water (HPLC quality) were then introduced with stirring. The mixture was then brought to 60° C. for 30 minutes with stirring, then cooled to RT.

| | | wt % of | | |
|---|---|---|---|---|
| Reactants | wt % | water | Amounts | Temperature (time) |
| 2-octyl cyanoacrylate | 10 | | 1 g | 60° C., 30 minutes |
| Cyclomethicone | 90 | | 9 g | |
| Water (HPLC) | | 78 | 36 g | |

### Example 2

### Synthesis of poly(2-octyl cyanoacrylate), Initiation by Anionic (Water) Route—Polymer P2

Introduced into a round-bottomed flask equipped with a magnetic stirrer bar were 0.5 g of 2-octyl cyanoacrylate, followed by 0.5 g of THF. The mixture was stirred for around 1 minute. 0.1 g of water (HPLC quality) was then introduced with stirring. The mixture was then brought to 60° C. for 30 minutes with stirring, then cooled to RT.

| | | wt % of | | |
|---|---|---|---|---|
| Reactants | wt % | water | Amounts | Temperature (time) |
| 2-octyl cyanoacrylate | 50 | | 0.5 g | 60° C., 30 minutes |
| Water | | 9 | 0.1 g | |
| THF | 50 | | 0.5 g | |

The polymer was obtained in solution in THF at a concentration of 50%.

### Example 2-1

Added to 1 g of the solution from Example 2 were 4.5 g of cyclomethicone, the THF was evaporated under vacuum on a rotary evaporator. The polymer P2 was obtained as a 10% dispersion in cyclomethicone.

### Example 3

Added to 2 g of polymer from Example 2 was a mixture of 1.3 g of α,ω-dihydroxylated polydimethylsiloxane and 7.7 g of cyclopentadimethylsiloxane. The THF was then evaporated. Thus, a 10% solution of the polymer P2 in the α,ω-dihydroxylated polydimethylsiloxane/cyclopentadimethylsiloxane mixture (14.7/85.3) was obtained.

### Example 4

### Synthesis of poly(2-octyl cyanoacrylate): Initiation by Radical (Peroxide) Route—Polymer P4

Introduced into a round-bottomed flask equipped with a magnetic stirrer bar were 0.5 g of methylheptyl cyanoacrylate, followed by 0.5 g of cyclomethicone. The mixture was stirred for around 1 minute. 25 mg of TRIGONOX 21S were added. The mixture was then brought to 75° C. for 3 hours with stirring, then cooled to RT.

| Reactants | wt % | Amounts | Temperature (time) |
|---|---|---|---|
| 2-octyl cyanoacrylate | 50 | 0.5 g | 75° C., 3 hours |
| Cyclomethicone | 50 | 0.5 g | |
| T21S | | 0.025 g | |

The formula was then diluted by cyclomethicone to obtain a 10% solution of polymer in cyclomethicone.

### Example 5

### Synthesis of poly(2-octyl cyanoacrylate): Initiation by Radical (Peroxide) Route—Polymer P5

Introduced into a round-bottomed flask equipped with a magnetic stirrer bar were 0.5 g of 2-octyl cyanoacrylate, followed by 0.5 g of THF. The mixture was stirred for around 1 minute. 25 mg of TRIGONOX 21S were added. The mixture was then brought to 75° C. for 3 hours with stirring, then cooled to RT. 4.5 g of D5 were then introduced to the mixture with vigorous stirring and the THF was distilled.

| Reactants | wt % | Amounts | Temperature (time) |
|---|---|---|---|
| 2-octyl cyanoacrylate | 50 | 0.5 g | 75° C., 3 hours |
| THF | 50 | 0.5 g | |
| T21S | | 0.025 g | |

### Example 6

### Synthesis of poly(2-octyl cyanoacrylate): Initiation by Radical (Persulphate) Route—Polymer P6

Introduced into a round-bottomed flask equipped with a magnetic stirrer bar were 0.2 g of methylheptyl cyanoacrylate, followed by 1.8 g of cyclomethicone. The mixture was stirred for around 1 minute. 20 mg of potassium persulphate (KPS), then 7.5 g of water were added. The mixture was then brought to 60° C. for 1 hour with stirring, then cooled to RT.

| | | wt % of | | |
|---|---|---|---|---|
| Reactants | wt % | water | Amounts | Temperature (time) |
| 2-octyl cyanoacrylate | 10 | | 0.2 g | 60° C., 1 hour |
| Cyclomethicone | 90 | | 1.8 g | |
| KPS | | | 0.020 g | |
| Water (pH = 3) | | 78 | 7.5 | |

### Example 7

### Synthesis of poly(butyl cyanoacrylate): Anionic Polymerization—Polymer P7

Introduced into a round-bottomed flask equipped with a magnetic stirrer bar were 1 g of butyl cyanoacrylate followed by 9 g of cyclomethicone (D5). The mixture was stirred for around 1 minute. 7.25 g of water (HPLC quality) were then introduced with stirring. The mixture was then brought to 60° C. for 30 minutes with stirring, then cooled to RT.

| | | wt % of | | |
|---|---|---|---|---|
| Reactants | wt % | water | Amounts | Temperature (time) |
| Butyl cyanoacrylate | 10 | | 1 g | 60° C., 30 minutes |
| Cyclomethicone | 90 | | 9 g | |
| Water (HPLC) | | 78 | 36 g | |

### Example 8

### Synthesis of poly(butyl cyanoacrylate): Anionic Polymerization—Polymer P8

Introduced into a round-bottomed flask equipped with a magnetic stirrer bar were 0.5 g of butyl cyanoacrylate, followed by 0.5 g of THF. The mixture was stirred for around 1 minute. 0.1 g of water (HPLC quality) was then introduced with stirring. The mixture was then brought to 60° C. for 30 minutes with stirring, then cooled to RT. The THF was evaporated under vacuum using a rotary evaporator. The product was dispersed in 4.5 g of cyclomethicone.

| | | wt % of | | |
|---|---|---|---|---|
| Reactants | wt % | water | Amounts | Temperature (time) |
| Butyl cyanoacrylate | 50 | | 0.5 g | 60° C., 30 minutes |
| Water | | 9 | 0.1 g | |
| THF | 50 | | 0.5 g | |

### Example 9

### Synthesis of poly(butyl cyanoacrylate): Initiation by Radical (Peroxide) Route—Polymer P9

Introduced into a round-bottomed flask equipped with a magnetic stirrer bar were 0.5 g of butyl cyanoacrylate, followed by 0.5 g of cyclomethicone. The mixture was stirred for around 1 minute. 25 mg of TRIGONOX 21S were added. The mixture was then brought to 75° C. for 3 hours with stirring, then cooled to RT.

| Reactants | wt % | Amounts | Temperature (time) |
|---|---|---|---|
| Butyl cyanoacrylate | 50 | 0.5 g | 75° C., 3 hours |
| Cyclomethicone | 50 | 0.5 g | |
| T21S | | 0.025 g | |

The formula was then diluted by cyclomethicone to obtain a 10% solution of polymer in cyclomethicone.

### Example 10

### Synthesis of poly(butyl cyanoacrylate): Initiation by Radical (Peroxide) Route—Polymer P10

Introduced into a round-bottomed flask equipped with a magnetic stirrer bar were 0.5 g of butyl cyanoacrylate, followed by 0.5 g of THF. The mixture was stirred for around 1 minute. 25 mg of TRIGONOX 21S were added. The mixture was then brought to 75° C. for 3 hours with stirring, then cooled to RT. 4.5 g of cyclomethicone were then introduced to the mixture with vigorous stirring and the THF was distilled.

| Reactants | wt % | Amounts | Temperature (time) |
|---|---|---|---|
| Butyl cyanoacrylate | 50 | 0.5 g | 75° C., 3 hours |
| THF | 50 | 0.5 g | |
| T21S | | 0.025 g | |

### Example 11

### Synthesis of poly(butyl cyanoacrylate): Initiation by Radical (Persulphate) Route—Polymer P11

Introduced into a round-bottomed flask equipped with a magnetic stirrer bar were 0.2 g of butyl cyanoacrylate, followed by 1.8 g of cyclomethicone. The mixture was stirred for around 1 minute. 20 mg of potassium persulphate (KPS), then 7.5 g of water were added. The mixture was then brought to 60° C. for 1 hour with stirring, then cooled to RT.

| | | wt % of | | |
|---|---|---|---|---|
| Reactants | wt % | water | Amounts | Temperature (time) |
| Butyl cyanoacrylate | 10 | | 0.2 g | 60° C., 1 hour |
| Cyclomethicone | 90 | | 1.8 g | |
| KPS | | | 0.020 g | |
| Water (pH = 3) | | 78 | 7.5 | |

### Example 12

### Synthesis of poly(methoxypropyl cyanoacrylate): Initiation by Anionic (Water) Route—Polymer P12

Introduced into a round-bottomed flask equipped with a magnetic stirrer bar were 1 g of methoxypropyl cyanoacrylate followed by 9 g of cyclomethicone (D5). The mixture was stirred for around 1 minute. 7.25 g of water (HPLC quality) were then introduced with stirring. The mixture was then brought to 60° C. for 30 minutes with stirring, then cooled to RT.

| | | wt % of | | |
|---|---|---|---|---|
| Reactants | wt % | water | Amounts | Temperature (time) |
| Methoxypropyl | 10 | | 1 g | 60° C., 30 minutes |
| cyanoacrylate | | | | |
| Cyclomethicone | 90 | | 9 g | |
| Water (HPLC) | | 78 | 36 g | |

### Example 13

### Synthesis of poly(methoxypropyl cyanoacrylate-co-methylheptyl cyanoacrylate) Copolymers by Anionic Route—Polymer P13

Introduced into a round-bottomed flask equipped with a magnetic stirrer bar were 0.5 g of methoxypropyl cyanoacrylate and 0.5 g of methylheptyl cyanoacrylate, followed by 9 g of cyclomethicone (D5). The mixture was stirred for around 1 minute. 7.25 g of water (HPLC quality) were then introduced with stirring. The mixture was then brought to 60° C. for 30 minutes with stirring, then cooled to RT.

| | | wt % of | | |
|---|---|---|---|---|
| Reactants | wt % | water | Amounts | Temperature (time) |
| Methoxypropyl | 5 | | 0.5 g | 60° C., 30 minutes |
| cyanoacrylate | | | | |
| 2-octyl cyanoacrylate | 5 | | 0.5 g | |
| Cyclomethicone | 90 | | 9 g | |
| Water (HPLC) | | 78 | 36 g | |

### Example 14

### Synthesis of poly(methoxypropyl cyanoacrylate-co-2-octyl cyanoacrylate) Copolymers by Radical (Peroxide) Route—Polymer P14

Introduced into a round-bottomed flask equipped with a magnetic stirrer bar were 0.25 g of methoxypropyl cyanoacrylate and 0.25 g of 2-octyl cyanoacrylate, followed by 0.5 g of cyclomethicone. The mixture was stirred for around 1 minute. 25 mg of Trigonox 21S were added. The mixture was then brought to 75° C. for 3 hours with stirring, then cooled to RT.

| Reactants | wt % | Amounts | Temperature (time) |
|---|---|---|---|
| Methoxypropyl | 25 | 0.25 g | 75° C., 3 hours |
| cyanoacrylate | | | |
| 2-octyl cyanoacrylate | 25 | 0.25 g | |
| Cyclomethicone | 50 | 0.5 g | |
| T21S | | 0.025 g | |

The formula was then diluted with cyclomethicone to obtain a 10% solution of polymer in cyclomethicone.

Applications to Hair of the Polymers from the Examples Above:

PROCEDURE 1: Deposit of polymer onto wet hair: 0.4 g of a composition comprising the polymer P1 to P14 having 10% of active material (AM) and cyclomethicone were applied to 2.7 g of wet natural hair and to 2.7 g of wet sensitized hair. The hair was mixed with the fingers for 1 to 2 minutes, then combed. The hair was then dried for 30 minutes under a hood dryer at 40° C.

Procedure 2: Deposit of Polymer onto Dry Hair

0.4 g of a composition comprising the polymer P1 to P14 having 10% of active material and cyclomethicone were applied to 2.7 g of natural and dry hair and to 2.7 g of sensitized and dry hair. The hair was mixed with the fingers for 1 to 2 minutes, then combed. The hair was then treated for 30 min under a hood dryer at 40° C.

### Procedure 3 (Control)

A control composition comprising 10 g of 2-octyl cyanoacrylate and 90 g of Dow Corning 245 Fluid was used for treating hair according to the same mode of application as that described for procedure 1 or 2. This composition did not contain polymerized octyl cyanoacrylate.

As a result of this treatment, the hair treated according to procedure 1 and 2 was coated, the locks were smoother, glossier and this coating lasted for at least 5 shampooings in comparison with the results obtained with procedure 3. In addition, a lasting volume and gloss of the hair was observed.

By scanning electron microscopy, before shampooing, a deposit having a “tubular” and even appearance was observed on natural hair and on sensitized hair. After 5 shampooings, the deposit was still present.

### Example 15

### Composition Comprising the Polymer and Monomer or Monomers+Polymerization of the Monomer by Anionic Route

0.4 g of a composition comprising the polymer P1 at a concentration of 5% AM in cyclomethicone and 5% AM of 2-octyl cyanoacrylate monomer in cyclomethicone was applied to 2.7 g of wet natural hair and 2.7 g of wet sensitized hair. After application according to procedure 1 described above, the hair was treated for 30 minutes under a hood dryer at 40° C.

RESULTS: The composition from Example 15 had a good affinity for the fibre and was evenly deposited. It remained in place on natural hair (5 shampooings).

### Example 16

### Deposits of 2 Polymers

A composition comprising P1 having 5% AM and P12 having 5% AM in cyclomethicone was applied according to the procedure from Example 15. After application according to procedure 1 described above, the hair was treated for 30 minutes under a hood dryer at 40° C.

RESULTS: The composition from Example 16 had a good affinity for the fibre and was evenly deposited. It remained in place on natural hair (5 shampooings).

1. Cosmetic composition for treating keratinous fibres, characterized in that it comprises at least one polymer soluble in the cosmetic composition and obtained from dielectrophilic monomers and optionally (meth)acrylic, (meth)acrylate or vinyl monomers other than the dielectrophilic monomers.

2. Cosmetic composition according to claim 1, characterized by the fact that the polymer is obtained from a monomer of formula (I): 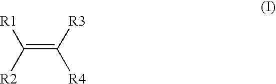
in which:
R₁ and R₂ each denote, independently of one another, a group that is not very or not at all electrophilic chosen from:
a hydrogen atom;
a linear, branched or cyclic, saturated or unsaturated hydrocarbon-based group preferably having from 1 to 20, better still from 1 to 10 carbon atoms, and optionally having one or more nitrogen, oxygen or sulphur atoms, and optionally substituted by one or more groups chosen from —OR, —COOR, —COR, —SH, —SR, —OH, and halogen atoms;
a modified or unmodified polyorganosiloxane residue; or
a polyoxyalkylene group;
R₃ and R4 each denote, independently of one another, an electrophilic group chosen from —N(R)₃⁺, —S(R)₂⁺, —SH₂⁺, —NH₃⁺, —NO₂, —SO₂R, —C≡N, —COOH, —COOR, —COSR, —CONH₂, CONHR, —F, —Cl, —Br, —I, —OR, —COR, —SH, —SR, —OH groups, linear or branched alkenyl groups, linear or branched alkynyl groups, C₁-C₄ monofluoroalkyl or polyfluoroalkyl groups, aryl groups such as a phenyl group, or aryloxy groups such as a phenoxyloxy group;
R denotes a linear, branched or cyclic, saturated or unsaturated hydrocarbon-based group preferably having from 1 to 20, better still from 1 to 10 carbon atoms, and optionally having one or more nitrogen, oxygen or sulphur atoms, and optionally substituted by one or more groups chosen from —OR′, —COOR′, —COR′, —SH, —SR′, —OH, halogen atoms, or a residue of a polymer which may be obtained by radical polymerization, by polycondensation or by ring opening, R′ denoting a C₁-C₁₀ alkyl group.

3. Composition according to claim 1 or 2, in which the polymer is obtained from dielectrophilic monomers chosen from monomers of formula (II): 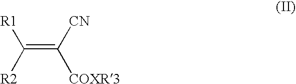
in which:
X denotes NH, S or O;
R₁ and R₂ each denote, independently of one another, a group with little or no electron-withdrawing effect chosen from:
a hydrogen atom;
a linear, branched or cyclic, saturated or unsaturated hydrocarbon-based group having from 1 to 20 carbon atoms, and optionally having one or more nitrogen, oxygen or sulphur atoms, and optionally substituted by one or more groups chosen from —OR, —COOR, —COR, —SH, —SR, —OH, and halogen atoms;
a modified or unmodified polyorganosiloxane residue; or
a polyoxyalkylene group;
R denotes a linear, branched or cyclic, saturated or unsaturated hydrocarbon-based group having from 1 to 20 carbon atoms, and optionally having one or more nitrogen, oxygen or sulphur atoms, and optionally substituted by one or more groups chosen from —OR′, —COOR′, —COR′, —SH, —SR′, —OH, halogen atoms, or a residue of a polymer which may be obtained by radical polymerization, by polycondensation or by ring opening, R′ denoting a C₁-C₁₀ alkyl group; and
R′₃ represents a hydrogen atom or a linear, branched or cyclic, saturated or unsaturated hydrocarbon-based group having from 1 to 20 carbon atoms, and optionally having one or more nitrogen, oxygen or sulphur atoms, and optionally substituted by one or more groups chosen from —OR′, —COOR′, —COR′, —SH, —SR′, —OH, halogen atoms, or a residue of a polymer which may be obtained by radical polymerization, by polycondensation or by ring opening, R′ denoting a C₁-C₁₀ alkyl group.

4. Cosmetic composition according to claim 2, characterized by the fact that the polymer is obtained from a monomer chosen from monomers in which X represents O.

5. Cosmetic composition according to any one of the preceding claims, in which the polymer is obtained from a dielectrophilic monomer chosen from ethyl 2-cyanoacrylate, methyl 2-cyanoacrylate, n-propyl 2-cyanoacrylate, isopropyl 2-cyanoacrylate, tert-butyl 2-cyanoacrylate, n-butyl 2-cyanoacrylate, isobutyl 2-cyanoacrylate, 3-methoxybutyl cyanoacrylate, n-decyl cyanoacrylate, hexyl 2-cyanoacrylate, 2-ethoxyethyl 2-cyanoacrylate, 2-methoxyethyl 2-cyanoacrylate, 2-octyl 2-cyanoacrylate, 2-propoxyethyl 2-cyanoacrylate, n-octyl 2-cyanoacrylate and iso-amyl cyanoacrylate, allyl 2-cyanoacrylate, methoxypropyl 2-cyanoacrylate.

6. Cosmetic composition according to claim 5, characterized by the fact that the polymer is obtained from a dielectrophilic monomer chosen from C₆-C₁₀ alkyl cyanoacrylates.

7. Cosmetic composition according to any one of the preceding claims, in which the polymer is obtained from octyl cyanoacrylate monomers of formula (V) and mixtures thereof: 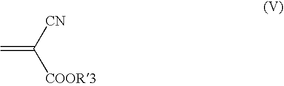
in which:
R′₃=—(CH₂)₇—CH₃;
—CH(CH₃)—(CH₂)₅—CH₃,
—CH₂—CH(C₂H₅)—(CH₂)₃—CH₃,
—(CH₂)₅—CH(CH₃)—CH₃ and
—(CH₂)₄—CH(C₂H₅)—CH₃.

8. Composition according to any one of the preceding claims, in which the polymer is obtained from at least 10% of dielectrophilic monomers.

9. Cosmetic composition according to any one of the preceding claims, characterized in that the polymer content is between 0.1 to 100% by weight of the total weight of the composition, preferably between 1 and 80% by weight of the total weight of the composition.

10. Cosmetic composition according to any one of the preceding claims, additionally comprising one or more polymerizable dielectrophilic monomers.

11. Composition according to claim 10, in which the dielectrophilic monomer is chosen from the monomers such as defined in the preceding claims 2 to 7.

12. Cosmetic composition according to any one of the preceding claims, comprising one or more mineral or organic pigments.

13. Cosmetic composition according to any one of the preceding claims, characterized by the fact that it comprises, in addition, at least one liquid organic solvent.

14. Cosmetic composition according to claim 13, characterized by the fact that the liquid organic solvent is chosen from aromatic alcohols, liquid fatty alcohols, modified or unmodified polyols, volatile silicones, liquid modified polydimethylsiloxanes, mineral, organic or vegetable oils, C₅ to C₁₀ alkanes, liquid fatty acids, liquid fatty esters and mixtures thereof.

15. Composition according to any one of the preceding claims, additionally comprising reducing agents, fatty substances, plasticizers, softeners, anti-foaming agents, moisturizers, clays, mineral fillers, UV screening agents, mineral colloids, peptizers, fragrances, preservatives, anionic, cationic, non-ionic or amphoteric surfactants, fixture or non-fixture polymers, polyols, proteins, vitamins, direct or oxidation dyes, pearling agents, propellants, and mineral or organic thickeners.

16. Method for the cosmetic treatment of keratinous fibres, characterized by the fact that the composition such as defined in any one of claims 1 to 15 is applied to said fibres.

17. Use of a cosmetic composition according to any one of claims 12, 13 or 14 for dyeing keratinous fibres.

18. Multi-compartment device or kit, comprising a first compartment, comprising a composition containing at least one polymerizable dielectrophilic monomer, and a second compartment, comprising at least one polymer obtained from one or more dielectrophilic monomers, the dielectrophilic monomers being such as defined in claims 2 to 7.
